(19) United States
(12) Patent Application Publication
(10) Pub. No.: US 2016/0025753 A1
(43) Pub. Date: Jan. 28, 2016

(54) ASSAY FOR CANNABINOIDS AND METHODS OF USE THEREOF

(71) Applicant: Microgenics Corporation, Fremont, CA (US)

(72) Inventors: Parisa Khosropour, Westlake Village, CA (US); Haijuan Li, Danville, CA (US); Concord Cheung, Fremont, CA (US); Anlong Ouyang, Fremont, CA (US); Lili Arabshahi, Oakland, CA (US)

(21) Appl. No.: 14/806,490

(22) Filed: Jul. 22, 2015

Related U.S. Application Data
(60) Provisional application No. 62/029,323, filed on Jul. 25, 2014.

(51) Int. Cl. G01N 33/94; G01N 33/543

(52) U.S. Cl. CPC G01N 33/948; G01N 33/54326; G01N 33/54306

(57) ABSTRACT

Immunoassays, methods, and kits for qualitative and/or quantitative detection of cannabinoids in specimens including, without limitation, bodily fluids (e.g., blood, urine, oral fluid or sweat) or other biological specimens and potential drug samples.

### CROSS-REFERENCE TO RELATED APPLICATIONS

This application claims the benefit of and priority to U.S. Prov. App. Ser. No. 62/029,323 filed 25 Jul. 2014, the entirety of which is incorporated herein by reference.

### BACKGROUND

Cannabinoids are a class of diverse chemical compounds. Cannabinoids are named for the cannabis plant, from which the first discovered cannabinoids were extracted. Certain cannabinoids activate cannabinoid receptors (CB1 and CB2) on cells that repress neurotransmitter release in the brain. These receptor proteins may be activated by endocannabinoids (produced naturally in the body by humans and animals), phytocannabinoids (found in cannabis and some other plants), and synthetic cannabinoids (produced chemically by humans).

The CB1 cannabinoid receptors are responsible for the psychoactive effects of cannabinoids and are primarily located in the brain. The CB2 cannabinoid receptors regulate the inflammatory process and are predominately located in the immune system. Both CB1 and CB2 cannabinoid receptors are G-protein-coupled receptors that are responsible for the mechanism of action of cannabinoid drugs via receptor-regulated transduction of the cyclic AMP system. The natural ligand of CB1 and CB2 is anandamide.

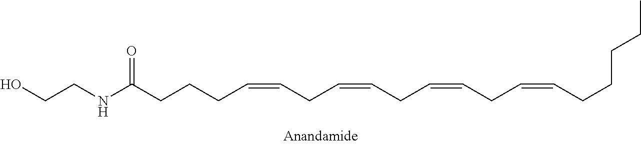 [SMILES: CCCCC/C=C\C/C=C\C/C=C\C/C=C\CCCC(=O)NCCO]

The primary psychoactive component of the cannabis plant is generally considered to be (−)-trans-Δ9-tetrahydrocannabinol (Δ9-THC). In the human body, Δ9-THC is mainly metabolized to 11-OH-THC, which is also psychoactive, and then further oxidized to 11-nor-Δ9-THC-9-COOH (THC-COOH) in the liver. Δ9-THC is shown below.

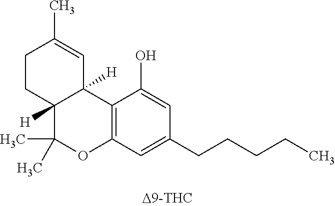 [SMILES: CCCCCc1cc(O)c2c(c1)OC(C)(C)[C@@H]1CCC(C)=C[C@@H]21]

Cannabidiol (CBD) is another major constituent of the plant, representing up to 40% in its extracts, but is non-psychotropic and has low affinity for CB1 and CB2 receptors. In total, approximately 85 different cannabinoids have been isolated from cannabis, exhibiting varied effects, although not all are psychoactive or otherwise biologically active.

In addition to natural and chemically-modified plant-derived cannabinoids, completely synthetic cannabinoids (also referred to as “cannabimimetics”) have been developed. Many of the synthetic cannabinoids are not based upon the dibenzopyran structure of Δ9-THC and, instead, form a diverse group of unrelated chemical structures with new synthetic cannabinoids continuously in development and appearing on the market. Synthetic cannabinoids encompass a variety of distinct chemical classes: the classical cannabinoids, which are structurally related to THC, and the nonclassical cannabinoids (cannabimimetics), which include naphthoylindoles, naphthylmethylindoles, naphthoylpyrroles, naphthylmethylindenes, phenylacetylindoles, and cyclohexylphenols (see, e.g., Table 1). Other cannabimimetics include, but are not limited to, aminoalkylindoles, 1,5-diarylpyrazoles, quinolines, and arylsulphonamides, as well as eicosanoids related to the endocannabinoids.

TABLE 1

| Structural Group of | |
|---|---|
| Synthetic | |
| Cannabinoid | Examples of Synthetic Cannabinoids |
| 1. Naphthoylindoles | 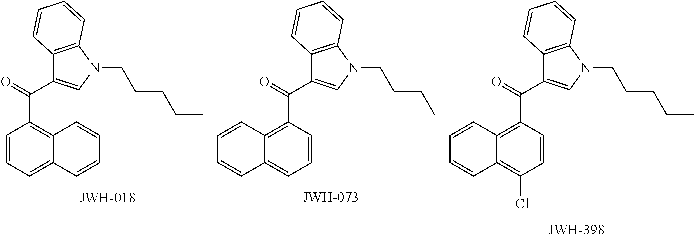 |
| 2. Naphthylmethylindoles | 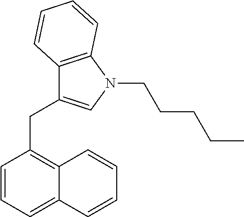 [SMILES: CCCCCn1cc(Cc2cccc3ccccc23)c2ccccc21] |
| | JWH-175 |
| 3. Naphthoylpyrroles | 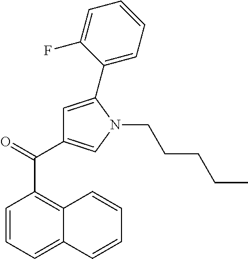 [SMILES: CCCCCn1cc(C(=O)c2cccc3ccccc23)cc1-c1ccccc1F] |
| | JWH-307 |
| 4. Naphthylmethylindenes | 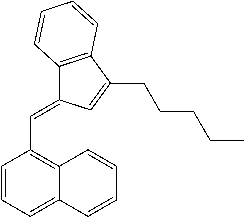 [SMILES: CCCCCC1=C/C(=C\c2cccc3ccccc23)c2ccccc21] |
| | JWH-176 |
| 5. Phenylacetylindoles | 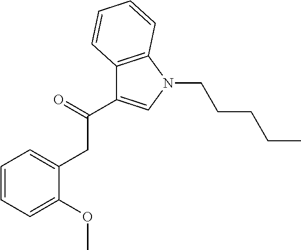 [SMILES: CCCCCn1cc(C(=O)Cc2ccccc2OC)c2ccccc21] |
| | JWH-250 |
| 6. Cyclohexylphenols | 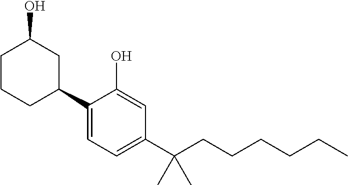 [SMILES: CCCCCCC(C)(C)c1ccc([C@H]2CCC[C@@H](O)C2)c(O)c1] |
| | C7-CP 47,497 |
| | 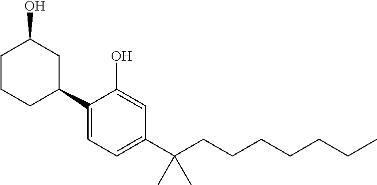 [SMILES: CCCCCCCC(C)(C)c1ccc([C@H]2CCC[C@@H](O)C2)c(O)c1] |
| | C8-CP 47,497 (cannabicyclohexanol) |
| 7. Classical cannabinoids | 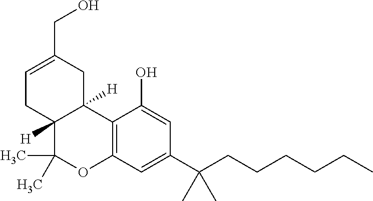 [SMILES: CCCCCCC(C)(C)c1cc(O)c2c(c1)OC(C)(C)[C@@H]1CC=C(CO)C[C@@H]21] |
| | HU-210 |

However, in spite of their diverse structures, essentially all biologically active (including psychoactive) cannabinoids share the common feature of being cannabinoid receptor agonists, capable of binding to CB1 and CB2, particularly the CB1 receptor. Psychoactive synthetic cannabinoids tend to be non-polar, lipid soluble and easily volatilized, making them ideal for addition to smoking mixtures. Studies have shown that at least some of the synthetic cannabinoids bind more strongly than Δ9-THC to CB1 receptors, increasing their potency, which may likewise increase the potential for abuse.

Due to structural dissimilarity between Δ9-THC and many synthetic cannabinoids, conventional drug screening methods via existing immunoassays developed for detection of Δ9-THC are ineffective in detecting the psychoactive synthetic cannabinoids. Currently, tests to detect synthetic cannabinoids rely upon more complex methods including gas chromatography-mass spectrometry (GC-MS) or liquid chromatography-tandem mass spectrometry (LC-MS/MS) methods but, in addition to being rather complex to perform, these methods are limited by the availability of pure reference materials. The need exists for an assay to detect a variety of different biologically active synthetic cannabinoids including assays which are sufficiently simple to perform and portable to allow for use outside of a laboratory setting, for example, by emergency medical responders. Further, the assays of the present invention may be useful as currently described even for the detection of biologically active synthetic cannabinoids not yet in existence.

### BRIEF SUMMARY

The present disclosure relates to assays, methods, and kits for qualitative and/or quantitative detection of cannabinoids (e.g., psychoactive and other biologically active cannabinoids). Such cannabinoids may be present in specimens including, without limitation, bodily fluids (e.g., blood, urine, oral fluid or sweat). Likewise, the assays described herein may be used for detecting cannabinoids in materials suspected of containing cannabinoids. For example, many synthetic cannabinoids are included in so-called incense products. Under conventional methods, many synthetic cannabinoids present in these samples and included in these products can avoid detection because the structure(s) of the cannabinoids are unknown to law enforcement and medical personnel. Nevertheless, the assays, methods, and kits described herein can be used to detect the presence of cannabinoids in these products. As used herein, “cannabinoids” is intended to indicate compounds that bind to one or both of the cannabinoid receptors CB1 and CB2 (e.g., psychoactive and other biologically active cannabinoids) including: (i) natural and chemically-modified plant-derived cannabinoids, (ii) completely synthetic cannabinoids (cannabimimetics), and (iii) derivatives of the above, unless otherwise indicated. Cannabinoids of diverse structure (e.g., natural or synthetic cannabinoids of known structure or synthetic cannabinoids that are unknown to law enforcement and medical personnel) can be detected using the assays described herein provided that the cannabinoid has some affinity for a cannabinoid receptor and/or is able to compete with a known cannabinoid for binding to a cannabinoid receptor.

In an embodiment, an assay for the presence of cannabinoid in a sample is described. The assay includes isolated cannabinoid receptor molecules, a reporter, and a sample suspected of containing an unknown cannabinoid. The presence of the unknown cannabinoid in the sample may be determined directly or indirectly by the reporter as a result of binding of the unknown cannabinoid to the isolated cannabinoid receptor molecules. In one embodiment, the reporter determines presence of the unknown cannabinoid in the sample by one or more of fluorescence, chemiluminescence, nephelometry, visually, or by agglutination (turbidimetry).

In another embodiment, a cannabinoid assay includes a sample suspected of containing an unknown cannabinoid, a capture reagent having one of isolated cannabinoid receptor molecules or a known cannabinoid bound to a solid phase, and a reporter configured to interact with the capture reagent to detect presence of the unknown cannabinoid. The presence of the unknown cannabinoid in the sample may be determined directly or indirectly as a result of binding of the unknown cannabinoid to the isolated cannabinoid receptor molecules.

In yet another embodiment, a kit for assaying for the presence of an unknown cannabinoid in a sample is described. The kit includes a first reagent that includes isolated cannabinoid receptor molecules, and a second reagent that includes a reporter capable of directly or indirectly detecting binding of an unknown cannabinoid to the isolated cannabinoid receptor molecules.

In still yet another embodiment, a method for detecting an unknown cannabinoid in a sample is disclosed. The method includes providing a sample suspected of containing an unknown cannabinoid, providing a first reagent that includes isolated cannabinoid receptor molecules and a second reagent that includes a reporter capable of directly or indirectly detecting binding of an unknown cannabinoid to the isolated cannabinoid receptor molecules, combining the sample with the first and second reagents, and detecting a presence or an amount of the unknown cannabinoid in the sample either directly or indirectly by detecting binding of the unknown cannabinoid to the isolated cannabinoid receptor molecules.

In the foregoing embodiments, the chemical structure of the unknown cannabinoid in the sample need not be known in order to detect its presence in the sample. Nevertheless, in some embodiments, the assays, kits, and methods described herein may include a known cannabinoid that competes with the unknown cannabinoid for binding to the isolated cannabinoid receptor molecules.

These and other objects and features of the present invention will become more fully apparent from the following description and appended claims, or may be learned by the practice of the invention as set forth hereinafter.

### BRIEF DESCRIPTION OF THE DRAWINGS

To further clarify the above and other advantages and features of the present invention, a more particular description of the invention will be rendered by reference to specific embodiments thereof which are illustrated in the appended drawings. It is appreciated that these drawings depict only illustrated embodiments of the invention and are therefore not to be considered limiting of its scope. The invention will be described and explained with additional specificity and detail through the use of the accompanying drawings in which:

FIG. 1 illustrates a schematic representation of a heterogeneous assay for detecting an unknown cannabinoid, according to one embodiment of the present disclosure.

FIG. 2 illustrates a schematic representation of another heterogeneous assay for detecting an unknown cannabinoid, according to one embodiment of the present disclosure.

FIG. 3 illustrates a schematic representation of another heterogeneous assay for detecting an unknown cannabinoid, according to one embodiment of the present disclosure.

FIG. 4 illustrates a schematic representation of a homogeneous assay for detecting an unknown cannabinoid, according to one embodiment of the present disclosure.

FIG. 5 illustrates a schematic representation of another homogeneous assay for detecting an unknown cannabinoid, according to one embodiment of the present disclosure.

FIG. 6 illustrates a schematic representation of another homogeneous assay for detecting an unknown cannabinoid, according to one embodiment of the present disclosure.

FIG. 7 illustrates a competitive inhibition dose-response curve according to the results of Example 1.

FIG. 8 is a bar graph illustrating the results from Example 2.

FIG. 9 illustrates a schematic representation of another heterogeneous assay for detecting an unknown cannabinoid, according to one embodiment of the present disclosure.

FIG. 10 illustrates a schematic representation of another heterogeneous assay for detecting an unknown cannabinoid, according to one embodiment of the present disclosure.

### DETAILED DESCRIPTION

The present disclosure relates to assays, methods, and kits for qualitative and/or quantitative detection of cannabinoids (e.g., psychoactive and other biologically active cannabinoids). Such cannabinoids may be present in specimens including, without limitation, bodily fluids (e.g., blood, urine, oral fluid or sweat). Likewise, the assays described herein may be used for detecting cannabinoids in materials suspected of containing cannabinoids. For example, many synthetic cannabinoids are included in so-called incense products. Under conventional methods, many synthetic cannabinoids present in these samples and included in these products can avoid detection because the structure(s) of the cannabinoids are unknown to law enforcement and medical personnel. Nevertheless, the assays, methods, and kits described herein can be used to detect the presence of cannabinoids in these products.

Cannabinoids

As used herein, the term “cannabinoids” is intended to indicate compounds that bind to one or both of the cannabinoid receptors CB1 and CB2 including: (i) natural and chemically-modified plant-derived cannabinoids, (ii) completely synthetic cannabinoids (cannabimimetics), and (iii) derivatives of the above, unless otherwise indicated. The “unknown cannabinoids” that are detected in the assays described herein may be structurally known cannabinoids (i.e., natural cannabinoids or known synthetic cannabinoids), but they are “unknown” in the sense that their presence may have not been detected or identified in a particular sample. Likewise, the “unknown cannabinoids” that are detected in the assays described herein can also be novel cannabinoids that are newly synthesized and that have not been identified. The presence of structurally novel cannabinoids can still be detected in the assays described herein even if their structure is unknown provided that they are able to bind to CB1 and/or CB2 or a derivative thereof (e.g., a CB1 or CB2 protein fragment) and/or if they are able to compete with a known cannabinoid for binding to a cannabinoid receptor. Generally, only biologically active (including psychoactive) cannabinoids will interact strongly with CB1 and CB2. Nevertheless, the biologically active cannabinoids are generally the cannabinoids that are of most interest to medical and law enforcement personnel.

Cannabinoid Receptors

Cannabinoid receptors are those that historically respond to cannabinoid drugs, such as THC and its analogs. Receptors themselves are membrane-bound G-protein coupled receptors (GPCR) found in many mammalian tissues and consists of 7 transmembrane domains (N-terminus is extracellular and C-terminus is cytoplasmic).

Two subtypes have been identified, CB1 and CB2. CB1 is 472 amino acids in length with a mass of 50-62 kDa (due to differing glycosylation) and is the product of the CNR1 gene. CB1 is mainly found in the nervous system, particularly in the brain. CB2 is 360 amino acids in length and is the product of the CNR2 gene. CB2 is found mainly in the immune system. Additional isoforms for both these subtypes have been observed which mainly changes that N-terminal sequence. Additional information about cannabinoid receptors can be found at http://www.iuphar-db.org/DATABASE/FamilyIntroductionForward?familyId=13. The assays described) herein utilize one or more of CB1, CB2, or derivatives or fragments thereof.

Assays

In an embodiment, an assay for the presence of cannabinoid in a sample is described. The assay includes isolated cannabinoid receptor molecules, a reporter, and a sample suspected of containing an unknown cannabinoid. The presence of the unknown cannabinoid in the sample may be determined directly or indirectly by the reporter as a result of binding of the unknown cannabinoid to the isolated cannabinoid receptor molecules. In one embodiment, the reporter determines presence of the unknown cannabinoid in the sample by one or more of fluorescence, chemiluminescence, nephelometry, visually, radiometry or turbidimetry (e.g., by agglutination). In one embodiment, the assay may include a known cannabinoid that competes with the unknown cannabinoid for binding to the isolated cannabinoid receptor molecules. In one embodiment, the assay may include a capture reagent having one of the isolated cannabinoid receptor molecules or a known cannabinoid bound to a solid phase such as, but not limited to, a microparticle, a magnetic microparticle, or a capture plate.

In certain embodiments the capture reagent(s) may include at least one solid phase having a plurality of addresses each of which has a distinct capture reagent disposed thereon (e.g., different cannabinoid receptor variants). In one embodiment the solid phase comprises a plurality of separately identifiable microparticles. In another embodiment the solid phase is a microarray.

Thus, to facilitate parallel processing (i.e., multiplexing) of large numbers of samples comprising unknown cannabinoids, the plurality of capture reagents are bound to at least one solid phase having a plurality of addresses each of which has a distinct, i.e., specific, capture reagent disposed thereon. The use of a solid phase is advantageous since it enables parallel processing of a large number of capture reagents whilst minimizing the time and effort required.

The solid phase may be any material known in the art, for example, a glass carrier, synthetic carrier, silicon wafer or membrane. Suitable materials include plastics, glasses, silicon, ceramics or organic polymers including polystyrene, polycarbonate, polypropylene, polyethylene, cellulose and nitrocellulose. The surface itself may be in the form, or part, of a slide, sheet, microplate or microtitre plate, tray, membrane, fibre, well, pellet, rod, stick, tube, bead and the like.

The term “address” is used to refer to a distinct feature of a solid phase or defined location on a solid phase that allows a specific capture reagent to be identified enabling the level of cannabinoid reactivity to that specific capture reagent to be determined.

In one embodiment the solid phase is a plurality of microparticles each being separately identifiable by means of an address. Suitable addresses include RFID tags, mass tags, fluorescent tags, optical encoding, digital magnetic tags, spectrometric encoding and the like.

As used herein, the term “reporter” refers to a substance that converts binding of the cannabinoid to the cannabinoid receptors to a detectable signal. Suitable example of means by which the reporter can determine the presence of the unknown cannabinoid in the sample includes, but is not limited to, one or more of fluorescence, chemiluminescence, nephelometry, visually, or by agglutination. Suitable examples of reporters include, but are not limited to, one or more of an anti-cannabinoid receptor antibody, an anti-cannabinoid receptor antibody/detection antibody pair, an agglutinator, or a labeled cannabinoid (i.e., a detection cannabinoid). Each of the reporters in the foregoing list will be discussed in greater detail below in reference to the various assay formats illustrated in the Figures.

Antibodies can be raised against the cannabinoid receptor using procedures that are well known in the art. In addition, there are a number of commercial antibodies available that are reactive to CB1 or CB2. For example, high quality anti-CB1 and anti-CB2 antibodies are commercially available from Thermo Fisher Scientific. Additional information about anti-CB1 antibody can be found at http://www.antibodypedia.com/gene_details.php?gene_id=3355. Additional information about anti-CB2 antibody can be found at http://www.antibodypedia.com/gene_details.php?gene_id=4047.

In one embodiment, the agglutinator used in the assay as a reporter may include at least one of an anti-cannabinoid receptor antibody, a carrier conjugated to multiple cannabinoid molecules, or a carrier conjugated to multiple isolated cannabinoid receptor molecules. In one embodiment, the purpose of the agglutinator is to join multiple components of the assay together so that binding of the cannabinoid by the cannabinoid receptor can be determined by, for example, a change in turbidity or a change in viscosity of the solution. In one embodiment, an anti-cannabinoid receptor antibody may be configured for agglutination of the isolated cannabinoid receptor molecules to one another.

In another embodiment, a cannabinoid assay includes a sample suspected of containing an unknown cannabinoid, a capture reagent having one of isolated cannabinoid receptor molecules or a known cannabinoid bound to a solid phase, and a reporter configured to interact with the capture reagent to detect presence of the unknown cannabinoid. The presence of the unknown cannabinoid in the sample may be determined directly or indirectly as a result of binding of the unknown cannabinoid to the isolated cannabinoid receptor molecules. In one embodiment, the solid phase is one of a microparticle, a magnetic microparticle, or a capture plate.

In one embodiment, the capture reagent may include a known cannabinoid bound to the solid phase, the isolated cannabinoid receptor molecules and the unknown cannabinoid may be present in a liquid phase, and the reporter may include an anti-cannabinoid receptor antibody. In this embodiment of the assay, the isolated cannabinoid receptor molecules may compete with the unknown cannabinoid for binding to the known cannabinoid of the capture reagent. In one embodiment, the anti-cannabinoid receptor antibody may be used with a secondary antibody (e.g., a detection antibody) that is reactive against the anti-cannabinoid receptor antibody. The secondary antibody may, for example, include a fluorescent dye or another detectable label that facilitates detection of the binding of the anti-cannabinoid receptor antibody to the cannabinoid receptor.

In one embodiment, the capture reagent comprises a known cannabinoid bound to the solid phase, the unknown cannabinoid is present in a liquid phase, and the reporter comprises a carrier conjugated to multiple isolated cannabinoid receptor molecules. In one aspect, the carrier conjugated to multiple isolated cannabinoid receptor molecules is capable of agglutinating the known cannabinoid bound to the solid phase for detection binding of the unknown cannabinoid to the isolated cannabinoid receptor molecules.

In one embodiment, the capture reagent comprises the isolated cannabinoid receptor molecules bound to the solid phase, the unknown cannabinoid is present in a liquid phase, and the reporter comprises an anti-cannabinoid receptor antibody. In one embodiment, the reporter further comprises a detection antibody capable of binding to the anti-cannabinoid receptor antibody.

In one embodiment, the capture reagent may include the isolated cannabinoid receptor molecules bound to the solid phase, the unknown cannabinoid is present in a liquid phase, and the reporter comprises a carrier conjugated to multiple known cannabinoid molecules. In one embodiment, the carrier conjugated to multiple known cannabinoid molecules is capable of agglutinating the cannabinoid receptor molecules bound to the solid phase for detection binding of the unknown cannabinoid to the isolated cannabinoid receptor molecules.

In one embodiment, the capture reagent may include known cannabinoid molecules (e.g., Δ9-THC) bound to the solid phase (e.g., one of a microparticle, a magnetic microparticle, or a capture plate), and the liquid phase includes (i) a sample suspected of containing an unknown cannabinoid and (ii) cannabinoid receptors (or fragment or derivative thereof) that may specifically bind the known cannabinoid to the unknown cannabinoid. In one embodiment, the known cannabinoid bound to the solid phase may compete with the unknown cannabinoid for capture of the cannabinoid receptor molecules. The presence of captured cannabinoid may be detected with the use of a labeled antibody or an antibody/labeled anti-antibody pair that binds to the cannabinoid receptor. Presence of the unknown cannabinoid is detected by a loss of signal from the assay relative to the amount of signal detected in the absence of the unknown cannabinoid.

In one embodiment, the capture reagent may include known cannabinoid molecules (e.g., Δ9-THC) bound to the solid phase (e.g., one of a microparticle, a magnetic microparticle, or a capture plate), and the liquid phase includes (i) a sample suspected of containing an unknown cannabinoid, (ii) a detection cannabinoid that is labeled with a detectable label, and (iii) cannabinoid receptor complexes (or fragments or derivatives thereof) that include at least two sites for simultaneous binding of at least two cannabinoids within each complex. The detection cannabinoid includes a detectable label—i.e., without limitation, any detectable label known in the art including visible dyes, fluorescent or chemiluminescent labels, radioisotopes and enzymes. In one embodiment, the known cannabinoid bound to the solid phase may capture cannabinoid receptor complexes having one or more available cannabinoid receptor binding sites. Presence of the unknown cannabinoid is detected by a loss of signal from the assay relative to the amount of signal detected in the absence of the unknown cannabinoid.

The assays described in the foregoing may also be included in kits. In one embodiment, a kit for assaying for the presence of an unknown cannabinoid in a sample is described. The kit includes a first reagent that includes isolated cannabinoid receptor molecules, and a second reagent that includes a reporter capable of directly or indirectly detecting binding of an unknown cannabinoid to the isolated cannabinoid receptor molecules.

In one embodiment, the reporter used in the kit may include one or more of an anti-cannabinoid receptor antibody, an anti-cannabinoid receptor antibody/detection antibody pair, an agglutinator, or a detection cannabinoid (i.e., a cannabinoid labeled with a detectable label).

In order to better describe the various assay formats and their components, reference will now be made to the specific assay formats illustrated in the Figures.

Heterogeneous Assays

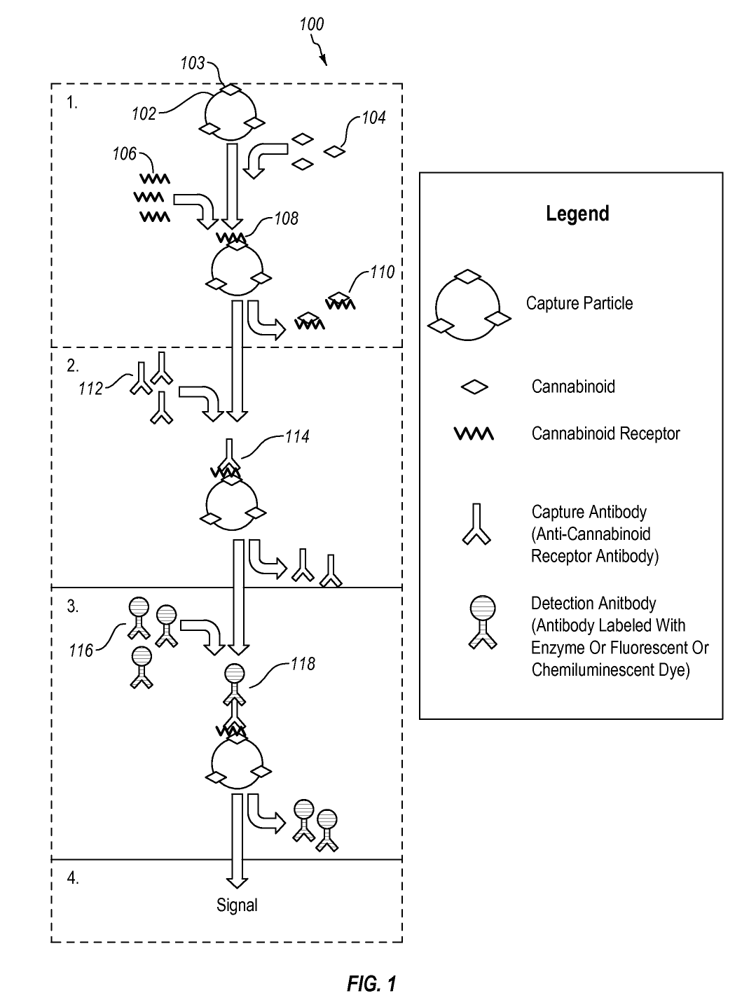

Referring to FIG. 1, a heterogeneous competitive assay 100 is illustrated. The heterogeneous competitive assay 100 is configured to detect the presence of an unknown cannabinoid having either a known or unknown structure in a sample by competing with a capture cannabinoid for binding to a soluble-phase cannabinoid receptor. Unbound cannabinoid receptor and cannabinoid receptor bound to the unknown cannabinoid is separated or washed away, and the remaining cannabinoid receptor bound to the capture cannabinoid may, for instance, be detected via labeled antibodies specific to the cannabinoid receptor structure or by an anti-cannabinoid receptor antibody/detection antibody pair. In the assay format illustrated in FIG. 1, the signal obtained is inversely related to the amount of the unknown cannabinoid present in the sample.

The assay 100 includes a solid support 102 having molecules of a cannabinoid or anandamide 103, or derivatives or mixtures thereof, attached to the solid surface 102. Molecules 103 may be termed the “capture” cannabinoid. Although the solid support is illustrated in FIG. 1 as comprising a plurality of magnetic particles, other types of solid supports may be used instead of magnetic particles, including without limitation plastic or glass plates, membranes, filters, or non-magnetic particles, as is known in the art. The solid support may be porous or non-porous.

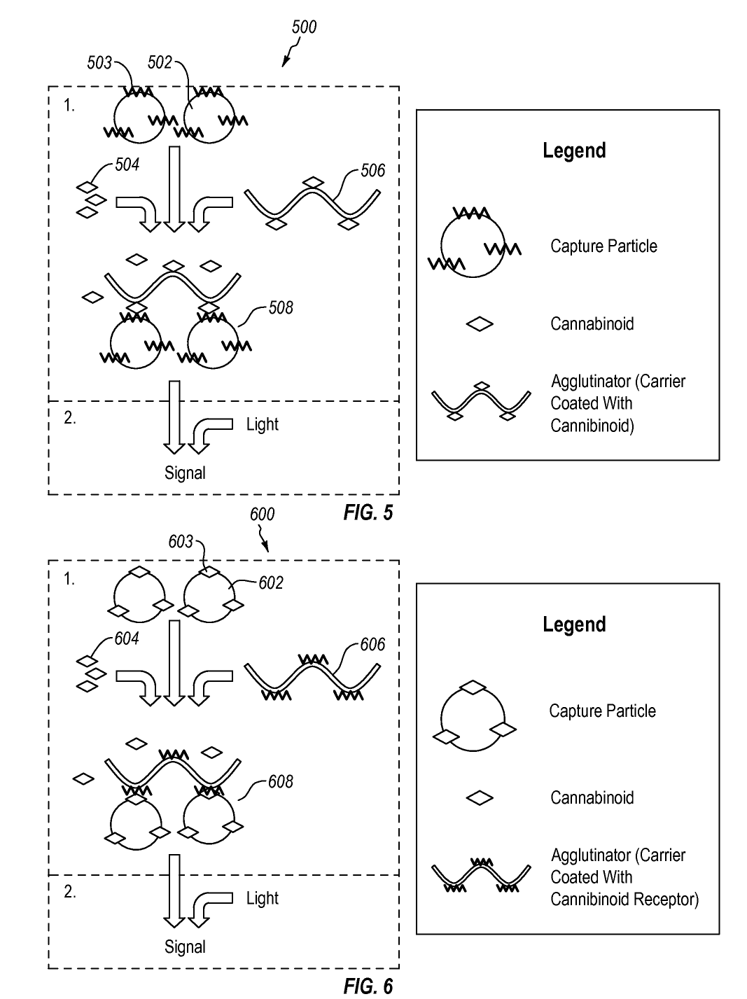

A sample suspected of containing cannabinoid molecules 104 is added to the solid support 102 along with a reagent comprising cannabinoid receptors 106, either simultaneously or sequentially. While cannabinoids 103 and 104 are both illustrated with diamond shapes in FIG. 1, this should not be taken to mean that cannabinoids 103 and 104 necessarily have the same structure. In some instances, the “capture” cannabinoid 103 and the unknown cannabinoid 104 may be the same (e.g., they may both be Δ9-THC), but this is not necessary. For instance, a known cannabinoid (e.g., Δ9-THC) may be used as cannabinoid 103 in an assay for the presence of a different type of cannabinoid 104 (e.g., a natural or synthetic cannabinoid). The forgoing is also true of cannabinoids 603, 604, 903, 904, 1003, and 1004 illustrated in FIGS. 6, 9 and 10 (see below).

One will also appreciate that the known cannabinoid 103 and the unknown cannabinoid 104 can each be single type of cannabinoid or they can be more than one type. For instance, there are numerous types of natural and synthetic cannabinoids with various affinities for cannabinoid receptor. An assay that includes multiple types of known cannabinoids 103 (i.e., capture cannabinoids) may be more sensitive than one that includes only a single type of capture cannabinoid. Likewise, drug screen samples and evidence samples may include more than one type of cannabinoid (e.g., a natural cannabinoid like Δ9-THC and a synthetic cannabinoid like cannabicyclohexanol.

As used herein throughout the specification, “cannabinoid receptors” means CB1 or CB2 or mixtures thereof. Preferably the receptor reagent comprises substantially CB1. Cannabinoids 104 (i.e., unknown cannabinoids) present in the sample compete with the cannabinoid molecules 103 bound to the solid support for binding to the cannabinoid receptors, as illustrated at 108. Unbound cannabinoid receptor molecules and receptor molecules bound to cannabinoid molecules present in the sample are separated from receptor molecules bound to cannabinoid molecules attached to the surface of the solid support as illustrated at 110. A first antibody 112 or mixture of antibodies capable of specifically binding to cannabinoid receptor is then added to the assay mixture. Binding of the antibody to the receptor is illustrated at 114.

After exposure to the first antibody 112 or mixture of antibodies, a labeled second antibody 116 that binds specifically to the anti-receptor antibodies 112 may be used. Alternatively, the first (anti-receptor) antibodies may be conjugated to a detectable label for detection of bound cannabinoid receptor (not shown in FIG. 1). Detectable labels include any known in the art including without limitation, visible dyes, fluorescent or chemiluminescent labels, radioisotopes and enzymes. As the concentration of the cannabinoid or synthetic cannabinoid in the sample tested increases, the amount of cannabinoid receptor bound to the solid support (and hence the amount of label detected) decreases, resulting in an inverse dose response curve with higher signal levels detected at lower levels of cannabinoid.

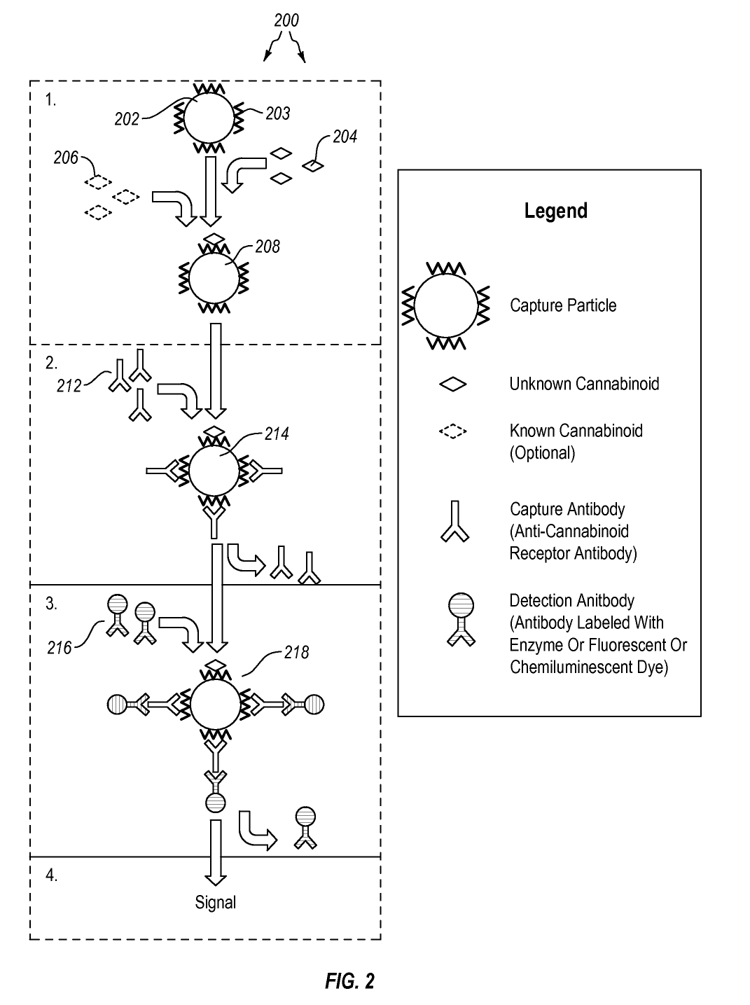

Referring now to FIG. 2, another heterogeneous competitive assay 200 is illustrated. Assay 200 is very similar to assay 100 except the solid support 202 includes isolated cannabinoid receptor molecules 203 instead of cannabinoids. Although the solid support is illustrated in FIG. 2 as comprising a plurality of magnetic particles, other types of solid supports may be used instead of magnetic particles, including without limitation plastic or glass plates, membranes, filters, or non-magnetic particles, as is known in the art. The solid support may be porous or non-porous.

A sample suspected of containing cannabinoid molecules 204 is added to the solid support 202. Optionally, a known cannabinoid 206 may also be added to the assay 200 in order to compete with the unknown cannabinoid 204. Cannabinoids 204 and 206 present in the assay compete with the cannabinoid molecules 203 bound to the solid support 202 for binding to the cannabinoid receptors 203, as illustrated at 208.

A first antibody 212 or mixture of antibodies capable of specifically binding to cannabinoid receptor is then added to the assay mixture. In the illustrated embodiment, antibody 212 is only able to bind to cannabinoid receptor that is not bound to a cannabinoid, as illustrated at 214.

After exposure to the first antibody 212 or mixture of antibodies, a labeled second antibody 216 that binds specifically to the anti-receptor antibodies 212 may be used. Alternatively, the first (anti-receptor) antibodies may be conjugated to a detectable label for detection of bound cannabinoid receptor (not shown in FIG. 2). Detectable labels include any known in the art including without limitation, visible dyes, fluorescent or chemiluminescent labels, radioisotopes and enzymes. As the concentration of the cannabinoid or synthetic cannabinoid in the sample tested increases, the amount of cannabinoid receptor bound to the solid support (and hence the amount of label detected) decreases, resulting in an inverse dose response curve with higher signal levels detected at lower levels of cannabinoid.

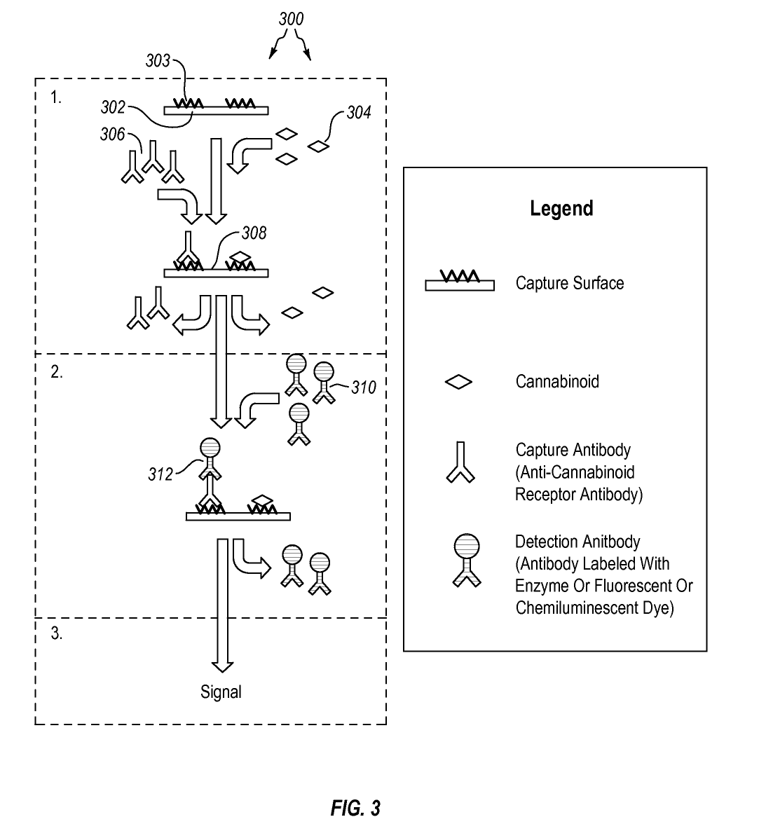

Another example of a heterogeneous competitive assay format 300 is illustrated in FIG. 3. The assay 300 includes a solid support 302 coated with cannabinoid receptor molecules (or fragment or derivative thereof) 303. The solid support 302 is then exposed to: (i) a sample suspected of containing cannabinoids 304 and (ii) a first antibody (or a mixture of antibodies) 306 that specifically bind the cannabinoid receptor (or fragment or derivative thereof). The first antibodies 306 compete with any cannabinoid molecules in the sample for binding to the cannabinoid receptors on the surface of the solid support, as illustrated at 308. After unbound anti-receptor antibodies and cannabinoids are removed, a second antibody 310 capable of binding the anti-receptor antibody, and which is conjugated to a detectable label, is added. Detectable labels include any known in the art including without limitation, visible dyes, fluorescent or chemiluminescent labels, radioisotopes and enzymes. Measurement of the signal generated by the detectable label results in an inverse dose response curve with a greater signal produced at lower levels of cannabinoid in the sample.

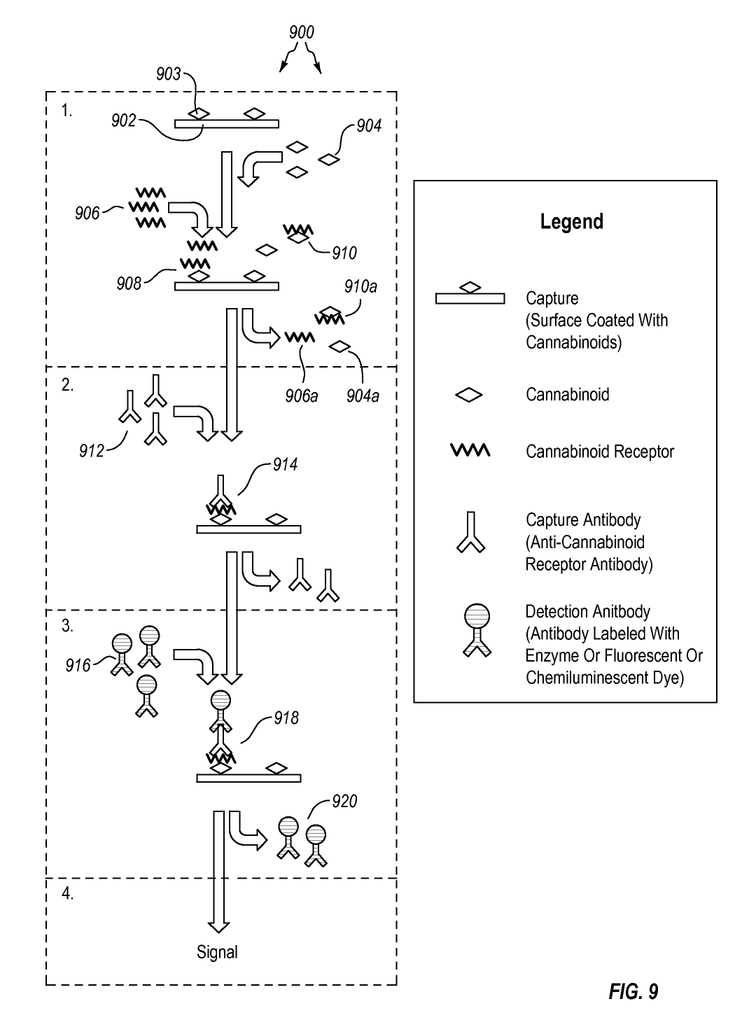

Another example of a heterogeneous competitive assay format 900 is illustrated in FIG. 9. The assay format illustrated in FIG. 9 is similar to the assay format of FIG. 1, except a solid surface (e.g., an assay plate) is used instead of the particle illustrated in FIG. 1. The assay 900 includes a solid support 902 (e.g., plastic or glass plates, membranes, filters, and the like) that is coated with a known cannabinoid (or fragment or derivative thereof) 903. The solid support 902 is then exposed to: (i) a sample suspected of containing an unknown cannabinoid 904 and (ii) cannabinoid receptors (or fragment or derivative thereof) 906 that specifically bind the known cannabinoid, as shown at 908, or to the unknown cannabinoid, as shown at 910. That is, the known cannabinoid 902 and the unknown cannabinoid 904 (if present) compete for binding with the cannabinoid receptor molecules 906. The amount of the cannabinoid receptor that is “pulled down” onto the known cannabinoid 903 bound to the surface is inversely related to the amount of unknown cannabinoid that is present in the sample. After allowing sufficient time for incubation, the receptor molecules bound to the unknown cannabinoid 910a, the unbound receptor molecules 906a, and the unbound unknown cannabinoid 904a are washed away. After the uncaptured material is washed away, an anti-cannabinoid receptor antibody 912 is added and allowed to bind to the cannabinoid receptor that was captured by the known cannabinoid, as illustrated at 914. After sufficient incubation time, the excess anti-cannabinoid receptor antibody 912 is washed away. Then, a detection antibody 916 that can bind to the anti-cannabinoid receptor antibody 912 is added and allowed to bind to the cannabinoid/receptor/antibody complex, as illustrated at 918. The detection antibody includes a detectable label—i.e., without limitation, any detectable label known in the art including visible dyes, fluorescent or chemiluminescent labels, radioisotopes and enzymes. After sufficient incubation time, excess detection antibody 916 is washed away (920) and the amount of detection antibody 916 captured is detected. Measurement of the signal generated by the detectable label results in an inverse dose response curve with a greater signal produced at lower levels of unknown cannabinoid 904 in the sample.

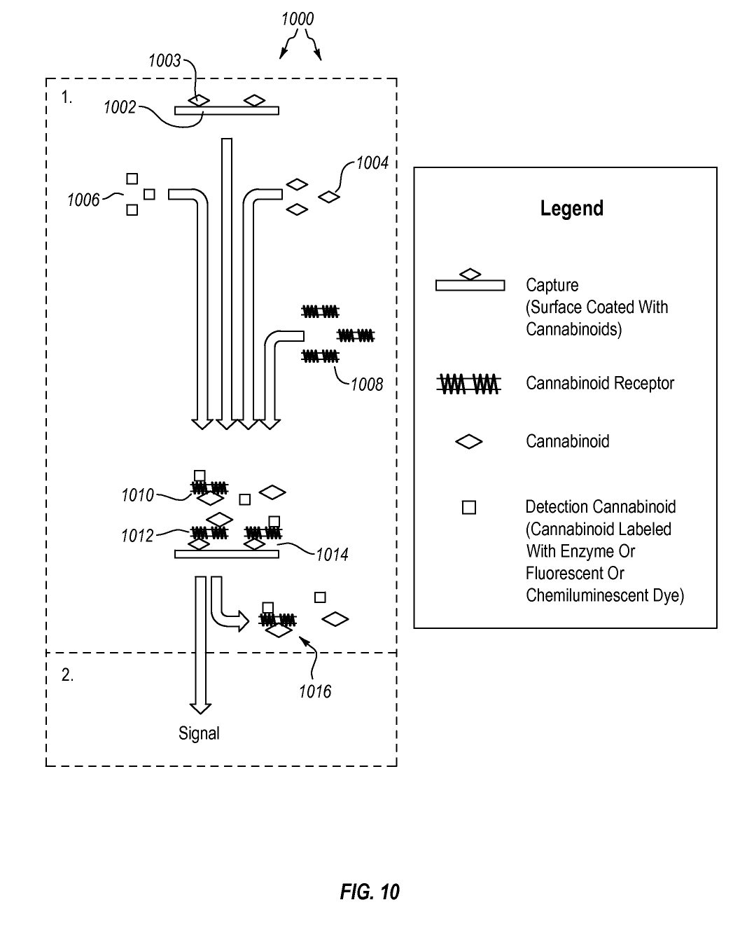

Another example of a heterogeneous competitive assay format 1000 is illustrated in FIG. 10. The assay 1000 includes a solid support 1002 (e.g., plastic or glass plates, membranes, filters, and the like) that is coated with a known cannabinoid (or fragment or derivative thereof) 1003. The soluble phase of the assay includes (i) a sample suspected of containing an unknown cannabinoid 1004, (ii) a detection cannabinoid 1006 that is labeled with a detectable label, and (iii) cannabinoid receptor complexes 1008 (or fragments or derivatives thereof) that include at least two sites for simultaneous binding of at least two cannabinoids within each complex. In the illustrated embodiment, the cannabinoid receptor complexes 1008 may be comprised of sections of disrupted membrane that include two or more membrane-bound cannabinoid receptors. Likewise, the cannabinoid receptor complexes 1008 may include cannabinoid receptor proteins (or fragments thereof) that are covalently linked to one another so that each complex 1008 includes two or more cannabinoid biding sites.

The assay of FIG. 10 is a competition assay similar to the other competition assays described herein, but with some notable differences. First, the competition is more complicated. The cannabinoid receptor complexes 1008 may bind to any combination of capture cannabinoid 1003, unknown cannabinoid 1004, or detection cannabinoid 1006. Cannabinoid receptor complexes 1008 may be captured by the capture cannabinoid 1003 if the complex 1008 includes a cannabinoid receptor site that is unbound to a solution-phase cannabinoid. This is illustrated schematically at 1012 and 1014. A receptor complex that cannot be captured, because it has no available cannabinoid receptor binding sites, is illustrated at 1010. Additionally, the unknown cannabinoid 1004 and the detection cannabinoid 1006 are in competition with one another. To illustrate this point, complex 1014 can be detected in the assay because complex 1014 includes a detection cannabinoid, while complex 1012 cannot be detected even though it is captured by the capture cannabinoid. The detection cannabinoid 1006 includes a detectable label—i.e., without limitation, any detectable label known in the art including visible dyes, fluorescent or chemiluminescent labels, radioisotopes and enzymes.

The assay 1000 includes adding (i) a sample suspected of containing an unknown cannabinoid 1004, (ii) a detection cannabinoid 1006 that is labeled with a detectable label, and (iii) cannabinoid receptor complexes 1008 to the solid support 1002 that is coated with the capture cannabinoid 1003 (i.e., the known cannabinoid (or fragment or derivative thereof)). After a sufficient incubation time, excess detection cannabinoid, unknown cannabinoid, and receptor complex are washed away, as illustrated at 1016. The amount of the detection cannabinoid that is “pulled down” onto the capture cannabinoid, via the cannabinoid receptor complex, is inversely related to the amount of detection cannabinoid and unknown cannabinoid that are present in the sample. Measurement of the signal generated by the detectable label results in an inverse dose response curve with a greater signal produced at lower levels of unknown cannabinoid 1004 in the sample.

Homogeneous Assays

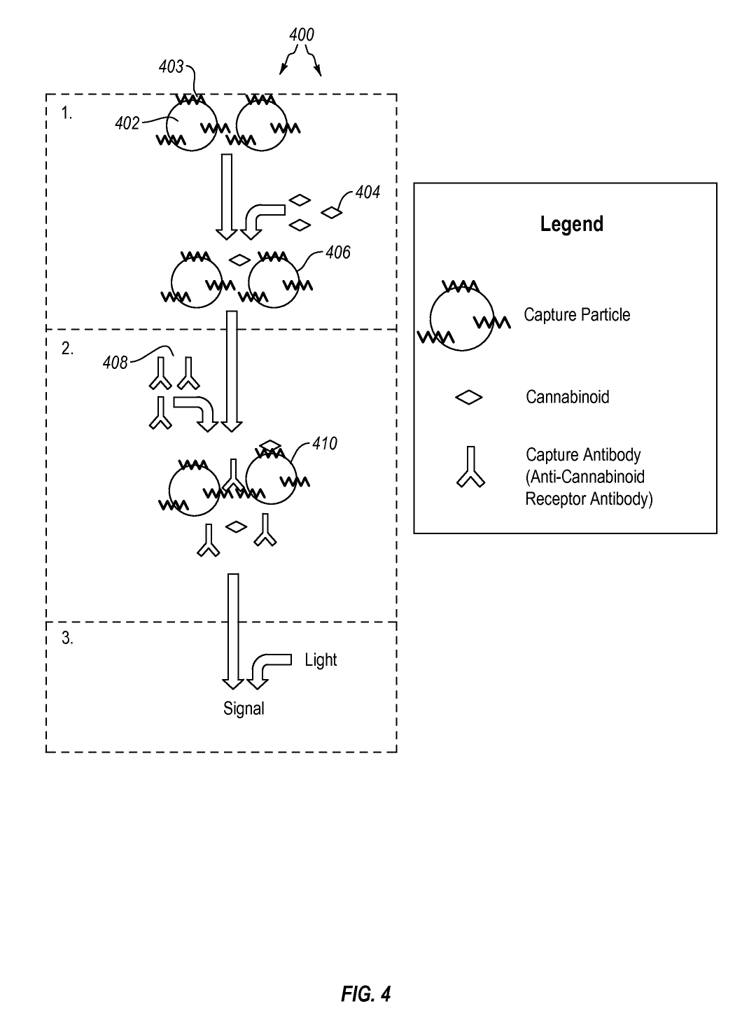

Referring now to FIG. 4, a homogeneous assay 400 format is illustrated. As shown in FIG. 4, a plurality of particles 402 are coated with cannabinoid receptor molecules 403. A sample suspected of containing cannabinoid molecules 404 is added to the particles 402 and, simultaneously or sequentially, an antibody or mixture of antibodies 408 capable of specifically binding cannabinoid receptor, is added. Cannabinoid molecules 404 in the sample, if present, will compete with antibodies 408 to cannabinoid receptor for binding to the receptor molecules bound to the surface of the particles 402. When little or no measurable level of cannabinoid is present in the sample, the particles are rapidly agglutinated by the addition of anti-receptor antibody, as illustrated at 410. But as levels of cannabinoid in a sample increase, the cannabinoid molecules compete with anti-receptor antibodies and inhibit, partially or completely, the agglutination reaction. Agglutination of the particles affects the absorbance of light and rate of absorbance change is measured photometrically. When a sample containing cannabinoid is added, the agglutination reaction is partially inhibited, slowing down the rate of absorbance change. A concentration-dependent agglutination inhibition curve can be obtained with the maximum rate of agglutination at the lowest cannabinoid concentration and the lowest agglutination rate at the highest cannabinoid concentration.

In an alternative homogeneous assay format 500, as depicted in FIG. 5, cannabinoid receptors molecules 503 are coated onto a plurality of particles 502. A reagent 506 comprising a carrier molecule conjugated to multiple molecules of cannabinoid or anandamide or mixtures thereof is used as an agglutination-inducing agent. As an example, the carrier molecule may comprise a polymer (such as dextran or proteins) and natural or synthetic membranes, fibers, and the like, as is known in the art. Cannabinoids 506 present in a sample compete with cannabinoids and/or anandamide bound to the agglutination-inducing agent for binding sites on the cannabinoid receptors bound to the particles. The particles rapidly agglutinate in the absence of any competing cannabinoid in a sample as illustrated at 508. The rate of absorbance change is measured photometrically. A concentration-dependent agglutination inhibition curve is obtained. The maximum rate of agglutination occurs at the lowest concentration of cannabinoid in the sample tested and the agglutination rate decreases as cannabinoid concentrations in the tested sample increase.

In another alternative homogeneous assay format, as depicted in FIG. 6, molecules of cannabinoids or anandamide or mixtures thereof 603 are coated onto a plurality of particles. A reagent 606 comprising multiple molecules of cannabinoid receptors coupled to a carrier (as described above) as an agglutination-inducing agent. Cannabinoids 604 present in a sample compete with cannabinoid and/or anandamide molecules bound to the particles for binding sites of cannabinoid receptors on the agglutination-inducing agent. The particles are rapidly agglutinated in the absence of any competing cannabinoid in a sample as illustrated at 608. The rate of absorbance change is measured photometrically. A concentration-dependent agglutination inhibition curve is obtained with the maximum rate of agglutination occurring at the lowest cannabinoid concentration in the sample tested and the lowest agglutination rate at the highest cannabinoid concentration.

The assay formats described in the forgoing may be used in methods for detecting an unknown cannabinoid in a sample suspected of containing a cannabinoid. In one embodiment, a method for detecting an unknown cannabinoid in a sample includes providing a sample suspected of containing an unknown cannabinoid, providing a first reagent that includes isolated cannabinoid receptor molecules and a second reagent that includes a reporter capable of directly or indirectly detecting binding of an unknown cannabinoid to the isolated cannabinoid receptor molecules, combining the sample with the first and second reagents, and detecting a presence or an amount of the unknown cannabinoid in the sample either directly or indirectly by detecting binding of the unknown cannabinoid to the isolated cannabinoid receptor molecules.

As described in greater detail in the foregoing reference to the Figures, detecting the presence or the amount of the unknown cannabinoid in the sample may include one or more of fluorescence, chemiluminescence, nephelometry, visually, or by agglutination.

In one embodiment, the method may further include providing a third reagent that includes a known cannabinoid that competes with the unknown cannabinoid for binding to the isolated cannabinoid receptor molecules.

Reference will now be made to specific examples that illustrate the assays and methods described herein.

### Example 1

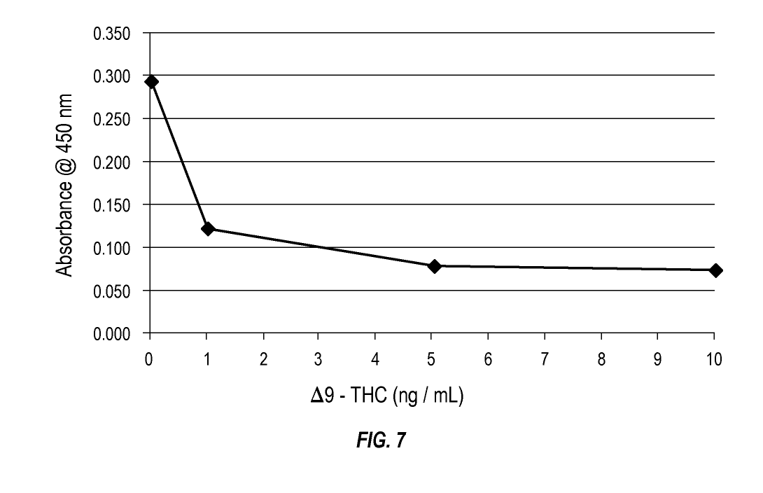

Paramagnetic particles (Sera-Mag carboxylate modified particles) were covalently coated with Δ9-THC conjugated to bovine serum albumin (BSA). The particles were prepared as a 2% solids solution in phosphate buffered saline (PBS), pH 7.4, with about 1% BSA. The cannabinoid receptor reagent comprised CB1 human recombinant protein (Abnova) solubilized in 25 mM Tris-HCl, pH 8.0, containing about 2% glycerol at a concentration of approximately 0.15 μg/mL. The cannabinoid test samples were prepared by spiking Δ9-THC in PBS solution, pH 7.4, with 0.05% Tween-20 non-ionic detergent. Dilutions of the samples ranging from 0 ng/mL to 10 ng/mL of Δ9-THC were tested. To 25 μL of particle solution, 33 μL of cannabinoid receptor reagent and 500 μL of the cannabinoid test sample were added. After incubation for approximately 2.5 hours at room temperature, the particles were separated from the reaction mixture via exposure to a magnetic force applied from outside the reaction vessel/tube. The reaction solution was removed and the particles were washed 3 times with PBS. After washing, the particles were suspended in 500 μL of antibody solution comprising affinity-purified rabbit polyclonal anti-CB1 antibody (EMD Millipore). After incubation for approximately 1.5 hour at room temperature, the particles were separated from the reaction mixture by magnetic separation. After washing, a 500 μL of secondary antibody solution containing goat anti-rabbit IgG labeled with horseradish peroxidase (Thermo Fisher Scientific) was added, followed by addition of the chromogenic substrate 3,3′,5,5′-Tetramethylbenzidine (TMB). The enzymatic reaction was stopped using sulfuric acid and the yellow reaction product was measured at 450 nm resulting in a competitive inhibition dose response curve with amounts of Δ9-THC as shown in FIG. 7.

### Example 2

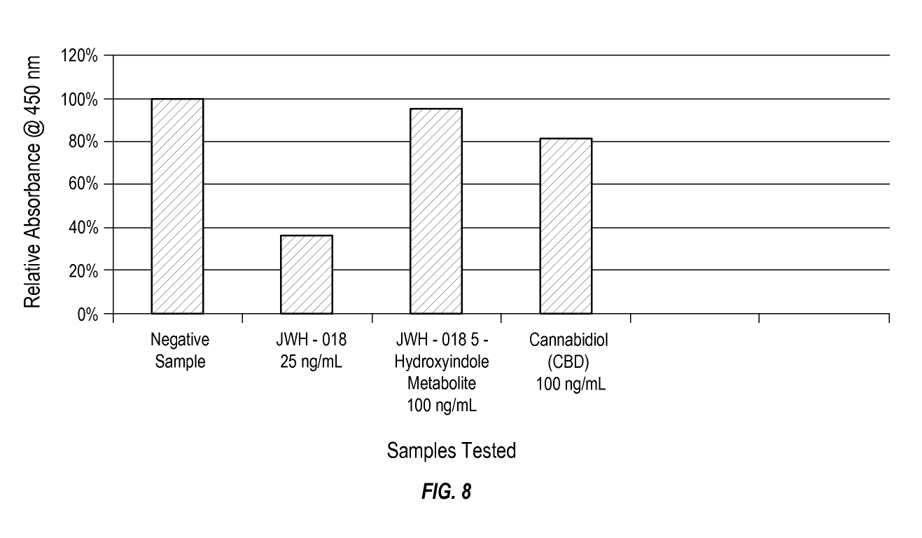

Paramagnetic particles (Sera-Mag carboxylate modified particles) were covalently coated with Δ9-THC conjugated to bovine serum albumin (BSA). A 2% solids particle solution was prepared in a phosphate buffered saline (PBS), pH 7.4, with approximately 1% BSA. The cannabinoid receptor reagent comprised CB1 human recombinant protein (Abnova) at a concentration of approximately 0.15 μg/mL solubilized in 25 mM Tris-HCl, pH 8.0, containing about 2% glycerol. Three test samples were prepared: 25 ng/mL of JWH-018, 100 ng/mL of JWH-018 5-hydroxyindole metabolite, and 100 ng/mL of CBD. In 25 μL of particle solution, 33 μL of cannabinoid receptor reagent and 500 μL of the cannabinoid test sample were added. After incubated for 3-4 hours at room temperature, the particles were separated from the reaction mixture by magnets. The reaction solution was removed and then the particles were washed 3 times with PBS. After washing, the particles were re-suspended in 500 μL of antibody solution containing affinity-purified rabbit polyclonal anti-CB1 antibody (EMD Millipore). After incubated for approximately 1.5 hours at room temperature, the particles were separated from the reaction mixture by magnets. After washing, a 500 μL of secondary antibody solution containing goat anti-rabbit IgG labeled with horseradish peroxidase (Thermo Fisher Scientific) was added. The chromogenic substrate 3,3′,5,5′-Tetramethylbenzidine (TMB) was added. The yellow reaction product was measured at 450 nm after the enzymatic reaction was stopped by sulfuric acid. The results in relative absorbance percentages are shown in FIG. 8. JWH-018 which is psychoactive demonstrated signal inhibition, while JWH-018 5-hydroxyindole metabolite and cannabidiol (each of which lacks significant psychoactivity) showed less signal inhibition.

### Example 3

Example 3 utilized an assay format similar to that illustrated in FIG. 9. The assay plate was prepared by incubating 0.5 μg of cannabinoid conjugates (Δ9-THC attached to a carrier protein) with each well of a 96-well microtiter plate overnight at 2-8° C. Wells were washed three times with 100 μL of phosphate buffered saline (PBS) with 0.05% Tween 20, pH 7.4. A 200 μL aliquot of a solution containing 2% bovine serum albumin (BSA) in PBS was added to each well and incubated at room temperature for 6 hours. The wells were washed for three times.

The assay was conducted with the prepared assay plate as follows: Either a blank sample or a sample containing 100 ng/ml of the cannabinoid analog HU-210 along with the CB1 human recombinant protein (Abnova) were added to the assay plate and incubated overnight at 2-8° C. After three rounds of washes, the antibody solution comprising 0.33 μg of affinity-purified rabbit polyclonal anti-cannabinoid receptor 1 antibody (EMD Millipore) was added and incubated for 6 hours at room temperature. The wells were washed for three times. Then, 0.05 mL of detection antibody, a goat anti-rabbit IgG labeled with horseradish peroxidase (HRP) (Thermo Fisher Scientific) was added and incubated for 1 hour at room temperature. After addition of the chromogenic substrate 3,3′,5,5′-tetramethylbenzidine (TMB) and incubation of 15 minutes, measurements at 450 nm resulted in significant inhibition of absorbance by the cannabinoid specimen containing HU-210. Results of the assay are illustrated below in Table 2.

TABLE 2

| | With 100 ng/mL | |
|---|---|---|
| Results | HU-210 | No Cannabinoid |
| Absorbance | 1.480 | 2.303 |
| @ 450 nm | 1.416 | 2.606 |
| | 1.602 | 2.288 |
| | 1.379 | 1.905 |
| | 1.569 | 2.350 |
| | 1.435 | 2.224 |
| Average | 1.480 | 2.279 |

A complex was formed via cannabinoid receptor by binding both cannabinoid conjugate adsorbed to the plate and capture antibody (anti-cannabinoid receptor antibody). Detection antibody labeled with HRP was bound to the cannabinoid receptor. The absorbance was generated by chromogenic substrate. With no cannabinoid present in the sample, the highest absorbance was detected. When a sample containing cannabinoid HU-210 was added, the absorbance was inhibited. Thus, the assay illustrates the inverse relationship between the presence of cannabinoid and absorbance that can be used for detecting or quantifying the cannabinoids in a sample. The assay results shown in Table 2 also illustrate that the response was repeatable and predictable across a number of replicates.

### Example 4

Example 3 utilized an assay format similar to that illustrated in FIG. 10. The assay plates were prepared manner similar to that described in Example 3. 0.5 μg of cannabinoid conjugates (Δ9-THC attached to a carrier protein) were incubated with each well of a 96-well microtiter plate overnight at 2-8° C. Wells were washed three times with 100 μL of phosphate buffered saline (PBS) with 0.05% Tween 20, pH 7.4. A 300 μL aliquot of a solution containing 2% bovine serum albumin (BSA) in PBS was added to each well and incubated at 2-8° C. overnight. The wells were washed for three times.

The assay was performed by adding to each well 25 μL of each cannabinoid sample, 25 μL of 0.01 mM fluorescent cannabinoid Tocrifluor T1117 (Tocris Bioscience) and human cannabinoid receptor 1 membrane preparation (EMD Millipore) diluted in 200 μl of incubation buffer containing 20 mM Tris-HCl, 104 mM NaCl, 0.00216 M KCl, 2.5 mM EDTA, 5 mM MgCl₂, 0.5 mg/mL BSA, pH 7.4. Then the plate was incubated at 30° C. for 90 minutes. After the incubation, each well was washed twice with cold incubation buffer and resuspended in 80 μL of incubation buffer. Fluorescence measurements were taken with excitation at 535 nm and emission at 610 nm. Results of the assay are illustrated below in Table 3.

TABLE 3

| | Relative Fluorescent Units |
|---|---|
| Sample | (RFU) |
| 1 μg/mL HU-210 | 21,014 |
| 1 μg/mL Δ9-THC | 21,834 |
| 1 μg/mL JWH-018 | 22,299 |
| 1 μg/mL Cannabicyclohexanol | 20,667 |
| No cannabinoids | 47,934 |

A complex was formed via cannabinoid receptor preparation with multiple cannabinoid binding sites (cannabinoid receptor membrane preparation) by binding both THC conjugate adsorbed to the plate and detection cannabinoid (cannabinoid labeled with a rhodamine dye). The fluorescence of the complex was measured. With no cannabinoid present in the sample, the higher fluorescent signal was detected. When a sample containing cannabinoid was added, the fluorescence was reduced. Thus, the inverse relationship between the presence of cannabinoid and fluorescence can be used for detecting or quantifying the cannabinoids in a sample. Likewise, the assay of Example 4 illustrates that the assay format is applicable to the detection of cannabinoids having a variety of different structures. For instance, classical cannabinoids Δ9-THC and HU-210 (box 7 of Table 1), JWH-018 (box 1 of Table 1), and cannabicyclohexanol (box 6 of table 1) are structurally diverse, yet the assay was able to detect all of them with a comparable response to a 1 μg/mL solution of each.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. An assay for the presence of cannabinoid in a sample, the assay comprising:
isolated cannabinoid receptor molecules;
a reporter; and
a sample suspected of containing an unknown cannabinoid,
wherein the presence of the unknown cannabinoid in the sample is determined directly or indirectly by the reporter as a result of binding of the unknown cannabinoid to the isolated cannabinoid receptor molecules.

2. The assay of claim 1, wherein a chemical structure of the unknown cannabinoid in the sample need not be known in order to detect its presence in the sample.

3. The assay of claim 1, further comprising a known cannabinoid that competes with the unknown cannabinoid for binding to the isolated cannabinoid receptor molecules.

4. The assay of claim 1, wherein the reporter determines presence of the unknown cannabinoid in the sample by one or more of fluorescence, chemiluminescence, spectrophotometry, spectroscopy, nephelometry, turbidimetry, visually, or radiometry.

5. The assay of claim 1, wherein the reporter comprises one or more of an anti-cannabinoid receptor antibody, an anti-cannabinoid receptor antibody/detection antibody pair, an agglutinator, or a labeled cannabinoid molecule.

6. The assay of claim 5, wherein the agglutinator comprises at least one of an anti-cannabinoid receptor antibody, a carrier conjugated to multiple cannabinoid molecules, or a carrier conjugated to multiple isolated cannabinoid receptor molecules.

7. The assay of claim 6, wherein the anti-cannabinoid receptor antibody is configured for agglutination of the isolated cannabinoid receptor molecules to one another.

8. The assay of claim 1, further comprising a capture reagent having one of the isolated cannabinoid receptor molecules or a known cannabinoid bound to a solid phase.

9. A cannabinoid assay, comprising:
a sample suspected of containing an unknown cannabinoid;
a capture reagent having one of isolated cannabinoid receptor molecules or a known cannabinoid bound to a solid phase; and
a reporter configured to interact with the capture reagent to detect presence of the unknown cannabinoid, wherein the presence of the unknown cannabinoid in the sample is determined directly or indirectly as a result of binding of the unknown cannabinoid to the isolated cannabinoid receptor molecules.

10. The cannabinoid assay of claim 9, wherein a chemical structure of the unknown cannabinoid in the sample need not be known in order to detect its presence in the sample.

11. The cannabinoid assay of claim 9, wherein the solid phase is one of a microparticle, a magnetic microparticle, a membrane, a filter, a filament, or a capture plate.

12. The cannabinoid assay of claim 9, wherein the capture reagent comprises a known cannabinoid bound to the solid phase, the isolated cannabinoid receptor molecules and the unknown cannabinoid are present in a liquid phase, and the reporter comprises an anti-cannabinoid receptor antibody.

13. The cannabinoid assay of claim 12, wherein the isolated cannabinoid receptor molecules compete with the unknown cannabinoid for binding to the known cannabinoid of the capture reagent.

14. The cannabinoid assay of claim 9, wherein the capture reagent comprises a known cannabinoid bound to the solid phase, the isolated cannabinoid receptor molecules, the unknown cannabinoid, and the reporter are present in a liquid phase, wherein the reporter comprises a known cannabinoid conjugated to a detectable label.

15. The cannabinoid assay of claim 14, wherein the isolated cannabinoid receptor molecules compete with the unknown cannabinoid and the reporter for binding to the known cannabinoid of the capture reagent.

16. The cannabinoid assay of claim 9, wherein the capture reagent comprises a known cannabinoid bound to the solid phase, the unknown cannabinoid is present in a liquid phase, and the reporter comprises a carrier conjugated to multiple isolated cannabinoid receptor molecules.

17. The cannabinoid assay of claim 16, wherein the carrier conjugated to multiple isolated cannabinoid receptor molecules is capable of agglutinating the known cannabinoid bound to the solid phase for detection binding of the unknown cannabinoid to the isolated cannabinoid receptor molecules.

18. The cannabinoid assay of claim 9, wherein the capture reagent comprises the isolated cannabinoid receptor molecules bound to the solid phase, the unknown cannabinoid is present in a liquid phase, and the reporter comprises an anti-cannabinoid receptor antibody.

19. The cannabinoid assay of claim 18, wherein the reporter further comprises a detection antibody capable of binding to the anti-cannabinoid receptor antibody.

20. The cannabinoid assay of claim 9, wherein the capture reagent comprises the isolated cannabinoid receptor molecules bound to the solid phase, the unknown cannabinoid is present in a liquid phase, and the reporter comprises a carrier conjugated to multiple known cannabinoid molecules.

21. The cannabinoid assay of claim 20, wherein the carrier conjugated to multiple known cannabinoid molecules is capable of agglutinating the cannabinoid receptor molecules bound to the solid phase for detection binding of the unknown cannabinoid to the isolated cannabinoid receptor molecules.

22. A kit for assaying for the presence of an unknown cannabinoid in a sample, the kit comprising:
a first reagent that includes isolated cannabinoid receptor molecules; and
a second reagent that includes a reporter capable of directly or indirectly detecting binding of an unknown cannabinoid to the isolated cannabinoid receptor molecules.

23. The kit of claim 21, wherein the reporter comprises one or more of an anti-cannabinoid receptor antibody, an anti-cannabinoid receptor antibody/detection antibody pair, an agglutinator, or a labeled cannabinoid.

24. A method for detecting an unknown cannabinoid in a sample, comprising:
providing a sample suspected of containing an unknown cannabinoid;
providing a first reagent that includes isolated cannabinoid receptor molecules and a second reagent that includes a reporter capable of directly or indirectly detecting binding of an unknown cannabinoid to the isolated cannabinoid receptor molecules;
combining the sample with the first and second reagents; and
detecting a presence or an amount of the unknown cannabinoid in the sample either directly or indirectly by detecting binding of the unknown cannabinoid to the isolated cannabinoid receptor molecules.

25. The method of claim 22, wherein detecting the presence or the amount of the unknown cannabinoid in the sample includes one or more of fluorescence, chemiluminescence, nephelometry, visual detection, or agglutination

26. The method of claim 22, further comprising providing a third reagent that includes a known cannabinoid that competes with the unknown cannabinoid for binding to the isolated cannabinoid receptor molecules.

27. The method of claim 22, wherein the reporter comprises one or more of an anti-cannabinoid receptor antibody, an anti-cannabinoid receptor antibody/detection antibody pair, an agglutinator, or a labeled cannabinoid.
